SEQUENCE LISTING (40 sequences)

SEQ ID NO: 1 - DNA, 22 bp, Artificial Sequence
  RT-PCR forward primer for S9 gene
gactccggaacaaacgtgaggt

SEQ ID NO: 2 - DNA, 20 bp, Artificial Sequence
  RT-PCR reverse primer for S9 gene
cttcatcttgccctcgtcca

SEQ ID NO: 3 - DNA, 20 bp, Artificial Sequence
  RT-PCR forward primer for CPT-1a gene
tccaccctgaggcatctatt

SEQ ID NO: 4 - DNA, 20 bp, Artificial Sequence
  RT-PCR reverse primer for CPT-1a gene
atgacctcctggcattctcc

SEQ ID NO: 5 - DNA, 20 bp, Artificial Sequence
  PR-PCR forward primer  for PPAR-alpha gene
agagccccatctgtcctctc

SEQ ID NO: 6 - DNA, 22 bp, Artificial Sequence
  RT-PCR reverse primer for PPAR-alpha gene
actggtagtctgcaaaaccaaa

SEQ ID NO: 7 - DNA, 20 bp, Artificial Sequence
  RT-PCR forward primer for ACOX gene
atgcctttgttgtccctatc

SEQ ID NO: 8 - DNA, 22 bp, Artificial Sequence
  RT-PCR reverse primer for ACOX gene
ccatcttcaggtagccattatc

SEQ ID NO: 9 - DNA, 20 bp, Artificial Sequence
  RT-PCR forward primer for SCD-1 gene
cgtctggaggaacatcattc

SEQ ID NO: 10 - DNA, 18 bp, Artificial Sequence
  RT-PCR reverse primer sequence for SCD-1 gene
agcgctggtcatgtagta

SEQ ID NO: 11 - DNA, 19 bp, Artificial Sequence
  RT-PCR forward primer  for MTTP gene
tctcacagtacccgttctt

SEQ ID NO: 12 - DNA, 21 bp, Artificial Sequence
  RT-PCR reverse primer for MTTP gene
tcttctccgagagacatatcc

SEQ ID NO: 13 - DNA, 22 bp, Artificial Sequence
  RT-PCR forward primer for ApoB gene
ggactgtctgacttccatattc

SEQ ID NO: 14 - DNA, 18 bp, Artificial Sequence
  RT-PCR reverse primer for ApoB gene
aagacttgccacccaaag

SEQ ID NO: 15 - DNA, 21 bp, Artificial Sequence
  RT-PCR forward primer for FAS gene
ctgcggaaacttcaggaaatg

SEQ ID NO: 16 - DNA, 20 bp, Artificial Sequence
  RT-PCR reverse primer for FAS gene
ggttcggaatgctatccagg

SEQ ID NO: 17 - DNA, 21 bp, Artificial Sequence
  RT-PCR forward primer for ACC-alpha gene
aggaggaccgcatttatcgac

SEQ ID NO: 18 - DNA, 19 bp, Artificial Sequence
  RT-PCR reverse primer for ACC-alpha gene
tgaccgtgggcacaaagtt

SEQ ID NO: 19 - DNA, 23 bp, Artificial Sequence
  RT-PCR forward primer for PPAR-gamma gene
aggccgagaaggagaagctgttg

SEQ ID NO: 20 - DNA, 22 bp, Artificial Sequence
  RT-PCR reverse primer for PPAR-gamma gene
tggccacctctttgctctgctc

SEQ ID NO: 21 - DNA, 21 bp, Artificial Sequence
  RT-PCR forward primer for SREBP-1c gene
gctaccggtcttctatcaatg

SEQ ID NO: 22 - DNA, 19 bp, Artificial Sequence
  RT-PCT reverse primer for SREBP-1c gene
gcaagaagcggatgtagtc

SEQ ID NO: 23 - DNA, 22 bp, Artificial Sequence
  RT-PCR forward primer for Caspase-2 gene
caatgctaactgtccaagtcta

SEQ ID NO: 24 - DNA, 19 bp, Artificial Sequence
  RT-PCR reverse primer for Caspace-2 gene
gggattgtgtgtggttctt

SEQ ID NO: 25 - DNA, 20 bp, Artificial Sequence
  RT-PCR forward primer for Leptin gene
tgacaccaaaaccctcatca

SEQ ID NO: 26 - DNA, 20 bp, Artificial Sequence
  RT-PCR reverse primer for Leptin gene
ccaggtcattggctatctgc

SEQ ID NO: 27 - DNA, 20 bp, Artificial Sequence
  RT-PCR forward primer for Adiponectin gene
tctccaggagtgccatctct

SEQ ID NO: 28 - DNA, 20 bp, Artificial Sequence
  RT-PCR reverse primer for Adiponectin gene
gttgcaagctctcctgttcc

SEQ ID NO: 29 - DNA, 18 bp, Artificial Sequence
  RT-PCR forward primer for Cyclin D1 gene
taggccctcagcctcact

SEQ ID NO: 30 - DNA, 20 bp, Artificial Sequence
  RT-PCR reverse primer for Cyclin D1 gene
ccacccctgggataaagcac

SEQ ID NO: 31 - DNA, 19 bp, Artificial Sequence
  RT-PCR forward primer for Cyclin D2 gene
cccgactcctaagacccat

SEQ ID NO: 32 - DNA, 22 bp, Artificial Sequence
  RT-PCR reverse primer for Cyclin D2 gene
ttcagcttacccaacactacca

SEQ ID NO: 33 - DNA, 18 bp, Artificial Sequence
  RT-PCR forward primer for Cyclin A2 gene
ggctgcaccaacagtaaa

SEQ ID NO: 34 - DNA, 21 bp, Artificial Sequence
  RT-PCR reverse primer for Cyclin A2 gene
gggtcagcatctatcaaactc

SEQ ID NO: 35 - DNA, 21 bp, Artificial Sequence
  RT-PCR forward primer for UCP-1 gene
aggcttccagtaccattaggt

SEQ ID NO: 36 - DNA, 22 bp, Artificial Sequence
  RT-PCR reverse primer for UCP-1 gene
ctgagtgaggcaaagctgattt

SEQ ID NO: 37 - DNA, 21 bp, Artificial Sequence
  RT-PCR forward primer for UCP-2 gene
atggttggtttcaaggccaca

SEQ ID NO: 38 - DNA, 22 bp, Artificial Sequence
  RT-PCR reverse primer for UCP-2 gene
cggtatccagagggaaagtgat

SEQ ID NO: 39 - DNA, 17 bp, Artificial Sequence
  RT-PCR forward primer of UCP-3 gene
cgaattggcctctacga

SEQ ID NO: 40 - DNA, 19 bp, Artificial Sequence
  RT-PCR reverse primer for UCP-3 gene
tgtaggcatccatagtccc

(19) United States
(12) Patent Application Publication
(10) Pub. No.: US 2016/0331801 A1
(43) Pub. Date: Nov. 17, 2016

(54) USE OF CASPASE-2 INHIBITORS TO TREAT AND PREVENT THE METABOLIC SYNDROME

(71) Applicant: Duke University, Durham, NC (US)

(72) Inventors: Anna Mae E. Diehl, Durham, NC (US); Gregory A. Michelotti, Chapel Hill, NC (US); Mariana V. Machado, Lisbon (PT); Sally A. Kornbluth, Durham, NC (US); Erika Seager-Johnson, Mebane, NC (US); Kelly R. Lindblom, Durham, NC (US)

(21) Appl. No.: 15/151,887

(22) Filed: May 11, 2016

Related U.S. Application Data
(60) Provisional application No. 62/159,508, filed on May 11, 2015.

(51) Int. Cl. A61K 38/08; C07K 16/40; A61K 38/05

(52) U.S. Cl. CPC A61K 38/08; A61K 38/05; C07K 16/40; C07K 2318/00; C07K 2317/76

(57) ABSTRACT

Methods and compositions for treatment of obesity, insulin resistance, hyperinsulinemia, type 2 diabetes mellitus, dyslipidemia, and nonalcoholic fatty liver disease are discloses. The methods and compositions relate to inhibition of caspase-2.

### CROSS-REFERENCE TO RELATED APPLICATIONS

This application is related to, claims priority to, and incorporated herein by reference for all purposes U.S. Provisional Patent Application No. 62/159,508, filed May 11, 2015.

### GOVERNMENT LICENSE RIGHTS

This invention was made with government support under DK0077794 and GM080333 awarded by the National Institutes of Health. The government has certain rights in this invention.

### BACKGROUND

### Obesity and NAFLD

The growing epidemics of obesity and its associated morbidities place obesity as a major health problem in the Western world. In the past 30 years, the prevalence of obesity has almost doubled worldwide. In fact, in 2008, 1.9 billion adults were overweight and half a billion obese, comprising 35% and 11% of the population (WHOM, 2013). In Europe, up to one fourth of the population is obese and in the United States, two thirds are overweight and one third obese (Nguyen and El-Serag, 2010; Tsigos et al., 2008).

Obesity associates with increased risk for insulin resistance (IR)/type 2 diabetes mellitus (T2DM), metabolic syndrome (MS), cardiovascular diseases and many cancers. MS is a cluster of factors that associate with IR and increased cardiovascular risk and is defined as the presence of at least three of five: abdominal obesity, hypertriglyceridemia, low high-density lipoprotein (HDL)-cholesterol levels, hypertension, and glucose intolerance (Grundy et al., 2004). Ectopic fat accumulation in the liver, nonalcoholic fatty liver disease (NAFLD), is strongly associated with the MS. In fact, NAFLD has been proposed to be both a component and a diagnostic criterion for the MS (Machado and Cortez-Pinto, 2014).

There is a great deal of interest in the metabolism of obesity-related diseases. Animal models and human studies in obesity have shown that visceral adipose tissue cannot cope with energy surplus. Adipocytes respond by hypertrophy, increasing in size and becoming dysfunctional, as evidenced by resistance to insulin and altered production adipokines (e.g., decreased expression of adiponectin and other anti-inflammatory and insulin sensitizing factors and increased expression of leptin and other factors that promote inflammation and insulin resistance) (Kloting and Bluher, 2014). The increased metabolic stress imposed upon adipocytes eventually triggers their death by apoptosis and paraptosis (Cinti et al., 2005). Adipocyte cell death has been described in mouse genetic and diet-induced obesity models, as well as humans with morbid obesity (Cinti et al., 2005; Strissel et al., 2007). Also, adipocyte cell size and death correlate with the presence of IR, MS and NAFLD (Cancello et al., 2005; Kloting and Bluher, 2014; Strissel et al., 2007). Cell death leads to release of fatty acids into the circulation; these fatty acids accumulate ectopically, inducing metabolic disturbances.

### Caspase-2

Caspase-2, an initiator caspase for apoptosis, is one of the first-identified caspases (Bouchier-Hayes and Green, 2012). Recently, caspase-2 expression and caspase-2 induced apoptosis have been associated with lipotoxicity. More specifically, intracellular accumulation of saturated fatty acids induced caspase-2 in both frog oocytes (Johnson et al., 2013) and hepatocytes in vitro, and caspase-2 was shown to be up-regulated in the livers of both humans and mice with NAFLD, a condition that increases hepatic accumulation of fatty acids (Machado et al., 2014). (Note: the Machado et al 2014 article is part of the disclosure of the present invention and is incorporated herein by reference.) Furthermore, it was shown that male rats fed a high fat diet exhibited increased mRNA expression of caspase-2 in retroperitoneal adipose tissue (Jobgen et al., 2009).

In the application below, we disclose that caspase-2 is the link between energy surplus and adipocyte death. Given that increased adipocyte death drives the development of the MS during obesity, our discovery identifies caspase-2 induction as the root cause of obesity-related MS. While evaluating the effects of caspase-2 deficiency in a mouse model of obesity caused by feeding energy-dense Western diets, we found that caspase-2 deletion had a dramatic protective effect in mice fed these obesogenic diets. Specifically, deleting caspase-2 prevented diet-induced adiposity, IR, dyslipidemia and NAFLD (i.e, the MS). These results identify caspase-2 as a novel therapeutic target for obesity, type 2 diabetes mellitus, and the MS. We propose the use of caspase-2 inhibitors as a treatment for nonalcoholic fatty liver disease (NAFLD) and other types of MS-related tissue damage.

### DESCRIPTION OF THE DRAWINGS

FIG. 1A. Caspase-2 deficient mice are protected from increased adiposity induced by Western diet. Right panel: Percentage increase in body weight in caspase-2 deficient mice and WT mice fed chow diet or Western diet. Left panel: Body weight at the end of treatment. Results graphed as mean±SEM. * P<0.05, ** P<0.01 chow versus Western diet; # P<0.05, ## P<0.01 WT versus caspase-2 deficient mice.

FIG. 1B. Caspase-2 deficient mice are protected from increased adiposity induced by Western diet. Percentage of body mass (DXA) and epididymal fat weight at the end of treatment. Results graphed as mean±SEM. * P<0.05, ** P<0.01 chow versus Western diet; # P<0.05, ## P<0.01 WT versus caspase-2 deficient mice.

FIG. 1C. Caspase-2 deficient mice are protected from increased adiposity induced by Western diet. Food efficiency analyzed from week 2 to week 15 of treatment; food ingested and high-corn syrup equivalent ingested during treatment. Results graphed as mean±SEM. * P<0.05, ** P<0.01 chow versus Western diet; # P<0.05, ## P<0.01 WT versus caspase-2 deficient mice.

FIG. 2A. Caspase-2 deficient mice are protected from type 2 diabetes mellitus induced by Western diet. Right panel: Glucose tolerance test. AUROC Western diet WT versus caspase-2 deficient mice: 190 versus 122, P=0.02. Left panel: Insulin tolerance test. AUROC 167 versus 118, P=0.0002. Results are graphed as mean±SEM. * P<0.05, ** P<0.01 chow versus Western diet; # P<0.05, ## P<0.01 WT versus caspase-2 deficient mice.

FIG. 2B. Caspase-2 deficient mice are protected from type 2 diabetes mellitus induced by Western diet. Fasting glycemia and HOMA. Results are graphed as mean±SEM. * P<0.05, ** P<0.01 chow versus Western diet; # P<0.05, ## P<0.01 WT versus caspase-2 deficient mice.

FIG. 2C. Caspase-2 deficient mice are protected from type 2 diabetes mellitus induced by Western diet. Size of pancreatic Langherhans islands. Results are graphed as mean±SEM. * P<0.05, ** P<0.01 chow versus Western diet; # P<0.05, ## P<0.01 WT versus caspase-2 deficient mice.

FIG. 2D. Caspase-2 deficient mice are protected from type 2 diabetes mellitus induced by Western diet. Representative sections stained with H&E (200 fold field) from pancreas.

FIG. 2E. Caspase-2 deficient mice are protected from type 2 diabetes mellitus induced by Western diet. Serum levels of total cholesterol, triglycerides and non-esterified fatty acids. Results are graphed as mean±SEM. * P<0.05, ** P<0.01 chow versus Western diet; # P<0.05, ## P<0.01 WT versus caspase-2 deficient mice.

FIG. 3A. Caspase-2 deficient mice are protected from NAFLD. Liver weight, liver-to-body weight ratio, % of liver fat (DXA) and liver triglyceride content in WT and caspase-2 deficient mice. Results are graphed as mean±SEM. * P<0.05, ** P<0.01 chow versus Western diet; # P<0.05, ## P<0.01 WT versus caspase-2 deficient mice.

FIG. 3B. Caspase-2 deficient mice are protected from NAFLD. Representative photos from Oil red staining in liver sections.

FIG. 3C. Caspase-2 deficient mice are protected from NAFLD. qRT-PCR analysis of liver RNA for genes involved in lipid metabolism, normalized to expression in chow-diet fed WT mice. Results are graphed as mean±SEM. * P<0.05, ** P<0.01 chow versus Western diet; # P<0.05, ## P<0.01 WT versus caspase-2 deficient mice.

FIG. 4A. Adipose tissue is fundamentally different in Caspase-2 deficient mice Leptin and adiponectin serum levels and qRT-PCR analysis in adipose tissue.

FIG. 4B. Adipose tissue is fundamentally different in Caspase-2 deficient mice Representative photos from H&E staining in adipose tissue sections and average size of adipocytes per 100× field.

FIG. 4C. Adipose tissue is fundamentally different in Caspase-2 deficient mice Number of adipocytes/HPF and cyclin expression (immunoblot and qRT-PCR).

FIG. 4D. Adipose tissue is fundamentally different in Caspase-2 deficient mice Proliferation (BrdU) in adipose tissue mesenchymal stem cells isolated from WT and caspace-2 deficient mice.

FIG. 4E. Adipose tissue is fundamentally different in Caspase-2 deficient mice Cleaved caspase-3 expression in adipose tissue (immunoblot and qRT-PCR).

FIG. 4F. Adipose tissue is fundamentally different in Caspase-2 deficient mice Uncoupling proteins expression (UCP) in adipose tissue (qRT-PCR and immunoblot). Results are graphed as mean±SEM and normalized to expression in chow-diet fed WT mice. * P<0.05, ** P<0.01 chow versus Western diet; # P<0.05, ## P<0.01 WT versus caspase-2 deficient mice.

FIG. 4G. Adipose tissue is fundamentally different in Caspase-2 deficient mice Right panel: oil red staining demonstrating 3T3-L1 cells differentiation in adipocytes. Left panel: apoptosis (caspase 3/7 activity) in adipocytes treated with Palmitate 1 mM, 48 hour±caspase-2 inhibitor, VDVAD-FMK 20 uM.

### DESCRIPTION OF INVENTION

By studying cohorts of wild-type versus caspase-2 deficient mice fed a western diet-type high-fat diet (n=8 mice/group), we have discovered a link between caspase-2, diet-induced obesity, and the metabolic syndrome. Compared to wild type control mice, caspase-2 deficient mice fed a western diet (high fat diet, enriched in saturated fatty acids and cholesterol, as well as with supplemented high fructose corn syrup equivalents in drinking water) are protected from diet-induced adiposity, insulin resistance and type 2 diabetes mellitus, dyslipidemia and nonalcoholic fatty liver disease (NAFLD). In summary, the mice are protected from the MS (metabolic syndrome). We propose the use of caspase-2 inhibitors in treating or preventing the MS, and its complications, including type 2 diabetes mellitus, dyslipidemia and NAFLD.

Additionally, we have discovered some mechanisms that may explain that protection. The adipose tissue from caspase-2 deficient mice is fundamentally different than that of wild type control mice. Specifically, caspase-2 deficiency results in increased numbers of adipocytes, increased adipocyte proliferative activity, reduced adipocyte apoptosis and decreased adipocyte size. Adipose expression of uncoupling proteins is increased by caspase-2 deficiency and correlates with differences in metabolic efficiency, suggesting that caspase-2-deficient adipocytes are able to oxidize fat more efficiently.

The aggregate data suggests that pharmacological inhibition of caspase-2 activity would improve adipose tissue abnormalities leading to improvement of metabolic disorders. Our data on 3T3L1 adipocytes supports this.

Caspase-2 is, therefore, a promising target for therapeutic inhibition in humans who are at risk for diet-induced obesity. Given that we observed no adverse effects of inhibiting caspase-2 in mice during short term obesogenic challenge (i.e. 16 weeks high fat diet), this deficiency is likely to be safe for longer durations. The latter possibility is supported by the fact that mice constitutively deficient in caspase-2 have a near normal phenotype, except for increased number of oocytes and 9% decrease in maximum lifespan (Zhang et al. 2007).

Also of note, despite deletion of an important apoptotic pathway, caspase-2 deficient mice do not develop spontaneous tumors. We believe that pharmacological inhibition of caspase-2 is a therapeutic tool to manage diet-induced obesity and the metabolic syndrome and its consequences, such as, insulin resistance/type 2 diabetes mellitus, dyslipidemia, cardiovascular disease and nonalcoholic fatty liver disease (and resultant cirrhosis and liver cancer). This would have tremendous implications for human health, since obesity is the pandemic of the modem world.

Thus, the present invention encompasses the following:

1). The use of caspase-2 inhibitors to treat and protect a patient from the typical outcomes that result from chronic ingestion of western diet, such as obesity, insulin resistance, beta-cell hyperplasia, type 2 diabetes mellitus, dyslipidemia, and nonalcoholic fatty liver disease.

2). The use of caspase-2 inhibitors as a method of reprogramming adipose tissue to decrease adipocyte size, increase adipocyte viability, increase adipocyte proliferation, increase adipocyte metabolic uncoupling and modulate gene expression in the liver.

### Methods of the Present Invention

In one embodiment, the present invention is the use of caspase-2 inhibitors to treat or protect a patient from the symptoms of the metabolic syndrome. In preferred embodiments, these symptoms are selected from the group consisting of obesity, insulin resistance, beta-cell hyperplasia, type 2 diabetes mellitus, dyslipidemia, and nonalcoholic fatty liver disease. In a most preferred embodiment of the invention, the symptom is nonalcoholic fatty liver disease.

One would first identify a typical patient. Typical criteria would include at least one of the following five conditions:

1. Overweight or obesity—BMI≧25 kg/m² with at least two conditions of the metabolic syndrome:

a). Increased waist circumference (≧102 if men and ≧89 cm if woman)

b). Serum triglycerides ≧150 mg/dL or under medication

c). HDL-cholesterol <40 mg/dL if men or 50 mg/dL if woman

d). Systolic blood pressure ≧130 mmHg, diastolic blood pressure ≧85 mmHg or under medication

e). Fasting glycemia ≧100 mg/dL

f). Non-alcoholic fatty liver disease (though not a classic criteria for metabolic syndrome)

2. Insulin resistance (HOMA≧3).

3. Dyslipidemia unresponsive to current standard of care

4. Increased cardiovascular risk (Framingham score >20 points in women and >12 points in men).

5. Non-alcoholic steatohepatitis with fibrosis ≧F2 (according to Brunt's staging)

One would then treat the patient with an effective amount of a caspase-2 inhibitor. Preferably, one would evaluate the inhibitor and determine the best mode of administration and most effective dose. In some embodiments, the dose will be given until symptoms are reduced. In other embodiments, the dose will be given indefinitely in order to prevent symptom reoccurrence or escalation. In some embodiments, the dose will be combined with other pharmaceuticals for treatment of multiple symptoms or treatment of one symptom with multiple drugs.

One would then follow up with the treated patients. For example:

a). Visits every months with anthropometric evaluation and blood pressure determination.

b). At the end of the first month—lab tests—CBC, liver profile, renal function, lipid profile, fasting glucose and insulin.

c). At the end of the sixth month—Lab tests: CBC, liver profile, renal function, lipid profile, fasting glucose and insulin, oral glucose tolerance test.

Treatment of a suitable patient with caspase-2 inhibitor is expected to result in improvement of at least one MS components. By “improvement” we mean that the MS will either regress to a more healthy profile or the symptom will not worsen as the case in an untreated patient. We also mean that the symptom may be prevented. Regarding NAFLD, we would expect normalization of aminotransferases and decrease in NAS score at least two points with no worsening/improvement of fibrosis after six months, and preferably one year, of treatment. In a preferred embodiment, one would find, as the primary endpoint, an improvement of NAS score at least 2 points, with improvement or no worsening on fibrosis of paired liver biopsy prior and post-treatment.

In another embodiment, the present invention is a method of reprogramming adipose tissue to decrease adipocyte size, increase adipocyte viability, increase adipocyte proliferation, increase adipocyte metabolic uncoupling or modulates gene expression in the liver comprising the step of treating a patient with an effective amount of caspase-2 inhibitor such that the patient's adipocytes are modified in a manner more consistent with a healthier individual. That would typically be evaluated indirectly by quantifying visceral fat (with magnetic resonance evaluation) and by measuring serum adipokines leptin and adiponectin, indicating restoration of a healthier adipocyte phenotype.

### Caspase-2 Inhibitors

The present invention requires the use of a caspase-2 inhibitor, preferably inhibitors that are specific for caspase-2. We provide a list of currently know inhibitors but note that the art will continue to develop and uncover new inhibitors suitable for the present invention.

Below please find a list of caspase-2 inhibitors:

1. IDN-6556 is a small molecule, broad-spectrum caspase inhibitor (pan-caspase inhibitor) with activity against all tested human caspases (including caspase-2). IDN-6556 shows no inhibition of other classes of proteases or other enzymes or receptors other than caspases. (Idun Pharmaceuticals.)

2. VDVAD is a cell-permeable, synthetic peptide that irreversibly inhibits the activity of caspase-2. It is available as a fluoromethylketone derivative that facilitates inhibition of cysteine proteases in a caspase-2-specific manner. A number of companies sell variations of this peptide inhibitor (for example, R&D Systems).

3. Monoclonal antibodies specific for caspase-2 are available (e.g., clone 691233; R&D Systems).

4. DARPin (i.e. Designed Ankyrin Repeat Proteins) is a genetically engineered antibody mimetic that has been described for caspase-2. (Schweizer, et al. 2007).

### Examples

### Experimental Procedures

### Animal Studies

Male wild-type (WT) mice C57B1/6 and caspase-2−/− (B6.1295Y-casp2tm1Yuan/J) were obtained from Jackson Laboratory (Bar Harbor, Me.). Mice were fed either chow diet (Picolab® Rodent diet 20, #5053; n=4 mice per genotype) or Western diet (TD.120330 22% HVO+0.2% cholesterol diet, Teklad Research, supplemented with fructose and glucose in the drinking water; n=8 mice per genotype) for 16 weeks, since 4 weeks of age. Diet specifications at Supplemental Table 1. At the end of treatment, mice were fasted for 8 hours and sacrificed. Animal care and procedures were approved by the Duke University Institutional Animal Care and fulfilled National Institutes for Health and Duke University IACUC requirements for humane animal care.

SUPPLEMENTAL TABLE 1
Characteristics of the diets

| | Chow diet | Western diet |
|---|---|---|
| Reference | Picolab© Rodent | TD.120330, 22% |
| | diet 20, #5053 | HVO + 0.2% cholesterol diet, |
| | | Teklad Research |
| Kcal/g of diet | 4.7 | 4.6 |
| % Fat as calories | 13 | 45.3 |
| % CH as calories | 62 | 37 |
| % Proteins as calories | 24 | 17.7 |

Glucose tolerance test was performed after 12 hours of fasting, with IP injection of glucose (2 g/kg). Glucose levels were measured at 0, 15, 30, 60, 90, 120 and 180 minutes, by tail vein sampling with portable glucometer. Insulin tolerance test was performed after 5 hours of fasting, with IP injection of 0.6 units/kg human regular insulin at concentration of 0.2 units/mL. Glucose levels were measured by tail vein sampling at 0, 15, 30, 45, and 60 minutes.

Histopathological, Serum and Tissue Analysis

Formalin-fixed, paraffin-embedded liver, pancreas and visceral (epididymal) adipose tissue biopsies were cut into 5 μm serial sections. H&E and Oil red staining were performed.

Insulin was measured with Ultrasensitive Mouse Insulin ELISA kit (Crystal Chem Inc: #90080). Lipids were measured with Triglyceride Colorimetric Assay kit (Cayman Chemical Company: #10010303), Free Fatty Acid Quantification Kit, (Abcam, ab65341) and Cholesterol Quantification kit (Abcam, ab65359); serum leptin and adiponectin were determined with Abcam mouse ELISA kits, ab100718 and ab108785, respectively.

Molecular Studies

mRNA quantification by Real-time Reverse Transcription-PCR (RT-PCR). Total RNA was extracted from livers or visceral adipose tissue using TRIzol (Invitrogen). RNA was reverse transcribed to cDNA templates using random primer and Super Script RNAse H-Reverse Transcriptase (Invitrogen) and amplified. Semiquantitative qRT-PCR was performed using iQ-SYBR Green Supermix (Bio-Rad) and StepOne Plus Real-Time PCR Platform (ABI/Life Technologies), as previously described (Michelotti et al., 2013). For primers, see Supplemental Table 2.

Supplemental Table 2-RT-PCR primers for analysis

| Gene | Primer forward | Primer reverse |
|---|---|---|
| S9 | GACTCCGGAACAAACGTGAGGT | CTTCATCTTGCCCTCGTCCA |
| | (SEQ ID NO: 1) | (SEQ ID NO: 2) |
| CPT-1a | TCCACCCTGAGGCATCTATT | ATGACCTCCTGGCATTCTCC |
| | (SEQ ID NO: 3) | (SEQ ID NO: 4) |
| PPAR-α | AGAGCCCCATCTGTCCTCTC | ACTGGTAGTCTGCAAAACCAAA |
| | (SEQ ID NO: 5) | (SEQ ID NO: 6) |
| ACOX | ATGCCTTTGTTGTCCCTATC | CCATCTTCAGGTAGCCATTATC |
| | (SEQ ID NO: 7) | (SEQ ID NO: 8) |
| SCD-1 | CGTCTGGAGGAACATCATTC | AGCGCTGGTCATGTAGTA |
| | (SEQ ID NO: 9) | (SEQ ID NO: 10) |
| MTTP | TCTCACAGTACCCGTTCTT | TCTTCTCCGAGAGACATATCC |
| | (SEQ ID NO: 11) | (SEQ ID NO: 12) |
| ApoB | GGACTGTCTGACTTCCATATTC | AAGACTTGCCACCCAAAG |
| | (SEQ ID NO: 13) | (SEQ ID NO: 14) |
| FAS | CTGCGGAAACTTCAGGAAATG | GGTTCGGAATGCTATCCAGG |
| | (SEQ ID NO: 15) | (SEQ ID NO: 16) |
| ACC-α | AGGAGGACCGCATTTATCGAC | TGACCGTGGGCACAAAGTT |
| | (SEQ ID NO: 17) | (SEQ ID NO: 18) |
| PPAR-γ | AGGCCGAGAAGGAGAAGCTGTTG | TGGCCACCTCTTTGCTCTGCTC |
| | (SEQ ID NO: 19) | (SEQ ID NO: 20) |
| SREBP-1c | GCTACCGGTCTTCTATCAATG | GCAAGAAGCGGATGTAGTC |
| | (SEQ ID NO: 21) | (SEQ ID NO: 22) |
| Caspase-2 | CAATGCTAACTGTCCAAGTCTA | GGGATTGTGTGTGGTTCTT |
| | (SEQ ID NO: 23) | (SEQ ID NO: 24) |
| Leptin | TGACACCAAAACCCTCATCA | CCAGGTCATTGGCTATCTGC |
| | (SEQ ID NO: 25) | (SEQ ID NO: 26) |
| Adiponectin | TCTCCAGGAGTGCCATCTCT | GTTGCAAGCTCTCCTGTTCC |
| | (SEQ ID NO: 27) | (SEQ ID NO: 28) |
| Cyclin D1 | TAGGCCCTCAGCCTCACT | CCACCCCTGGGATAAAGCAC |
| | (SEQ ID NO: 29) | (SEQ ID NO: 30) |
| Cyclin D2 | CCCGACTCCTAAGACCCAT | TTCAGCTTACCCAACACTACCA |
| | (SEQ ID NO: 31) | (SEQ ID NO: 32) |
| Cyclin A2 | GGCTGCACCAACAGTAAA | GGGTCAGCATCTATCAAACTC |
| | (SEQ ID NO: 33) | (SEQ ID NO: 34) |
| UCP-1 | AGGCTTCCAGTACCATTAGGT | CTGAGTGAGGCAAAGCTGATTT |
| | (SEQ ID NO: 35) | (SEQ ID NO: 36) |
| UCP-2 | ATGGTTGGTTTCAAGGCCACA | CGGTATCCAGAGGGAAAGTGAT |
| | (SEQ ID NO: 37) | (SEQ ID NO: 38) |
| UCP-3 | CGAATTGGCCTCTACGA | TGTAGGCATCCATAGTCCC |
| | (SEQ ID NO: 39) | (SEQ ID NO: 40) |

CPT1a, Carnitine Palmitoyltransferase 1A; PPAR-α, Peroxisome Proliferator-Activated Receptor-α; ACOX, Acyl Coenzyme A Oxidase; SCD-1, Stearoyl-CoA Desaturase; MTTP, Microsomal Triglyceride Transfer Protein; ApoB, Apolipoprotein B; FAS, Fatty Acid Synthase; ACC-α, Acetyl-CoA Carboxylase-α; PPAR-γ, Peroxisome Proliferator-Activated Receptor-γ; SREBP-1c, Sterol Regulatory Element Binding Protein-1c; UCP, uncoupling protein.

Western Blotting

Total proteins were extracted from visceral adipose tissue using RIPA buffer (Sigma) supplemented with phosphatase and protease inhibitors (Roche). Equal amounts of protein were separated by electrophoresis on 4%-20% Criterion gels (BioRad), transblotted into polyvinylidene difluoride membranes, and incubated with primary antibodies listed in Supplemental Table 3.

Cell Isolation and Culture

SUPPLEMENTAL TABLE 3
Primary Antibodies for Western Blot

| | | | Catalog | |
|---|---|---|---|---|
| Antibody | Host | Company | Number | Dilution |
| Cyclin A | Rabbit | Santa Cruz | Sc-596 | 1:500 |
| | | Biotechnology | | |
| UCP-2 | Goat | Santa Cruz | Sc-6525 | 1:500 |
| | | Biotechnology | | |
| Cleaved Caspase-3 | Rabbit | Cell Signaling | #9661 | 1:500 |
| | | Technology | | |
| α-Tubulin | Mouse | Abcam | Ab4074 | 1:5000 |

LC3, microtubule-associated protein 1A/1b-light chain 3.

Mouse adipose tissue stem cells (ASC) were isolated from 4 caspase-2−/− and 4 WTmice as previously described (Fink and Zachar, 2011). BrdU assay (Cell Signaling) were performed according to manufacturer indications.

3T3-L1 cells were differentiated in adipocytes as previously described (Zebisch et al., 2012), and differentiation confirmed with Oil red staining. Adipocytes were treated with palmitate 1 mM with or without caspase-2 inhibitor Z-VDVAD-FMK (R&D Systems), 20 uM, for 48 hours. Apoptosis was measured using ApoTox-Glo Triplex Assay (Promega).

Statistics

Results were expressed as mean±SEM. Significance was established using T-student and Mann-Whitney tests, with significance p<0.05.

Results

Caspase-2 Deficient Mice are Protected from Diet-Induced Obesity

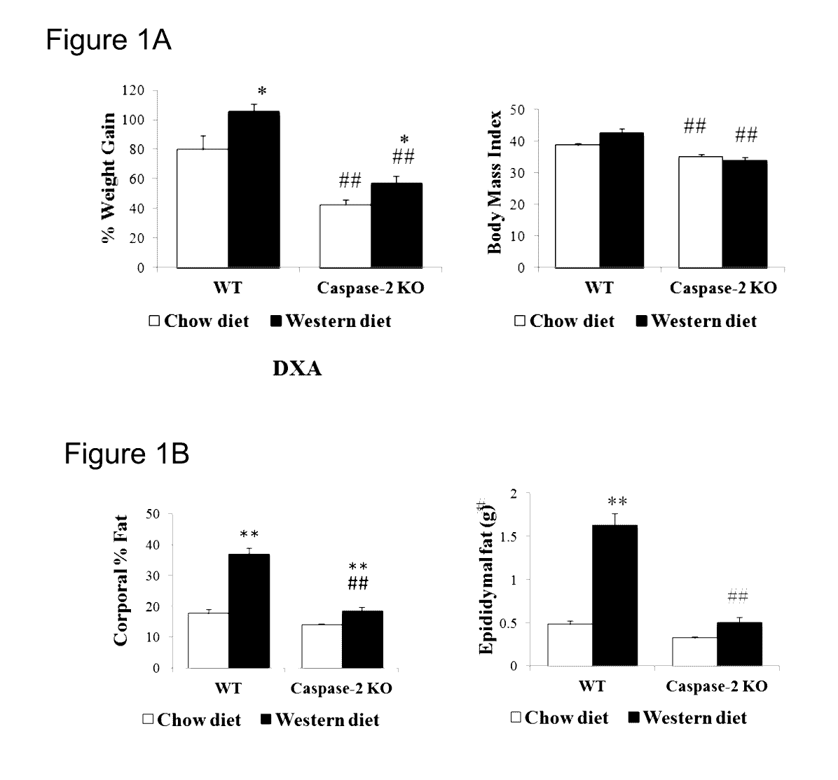

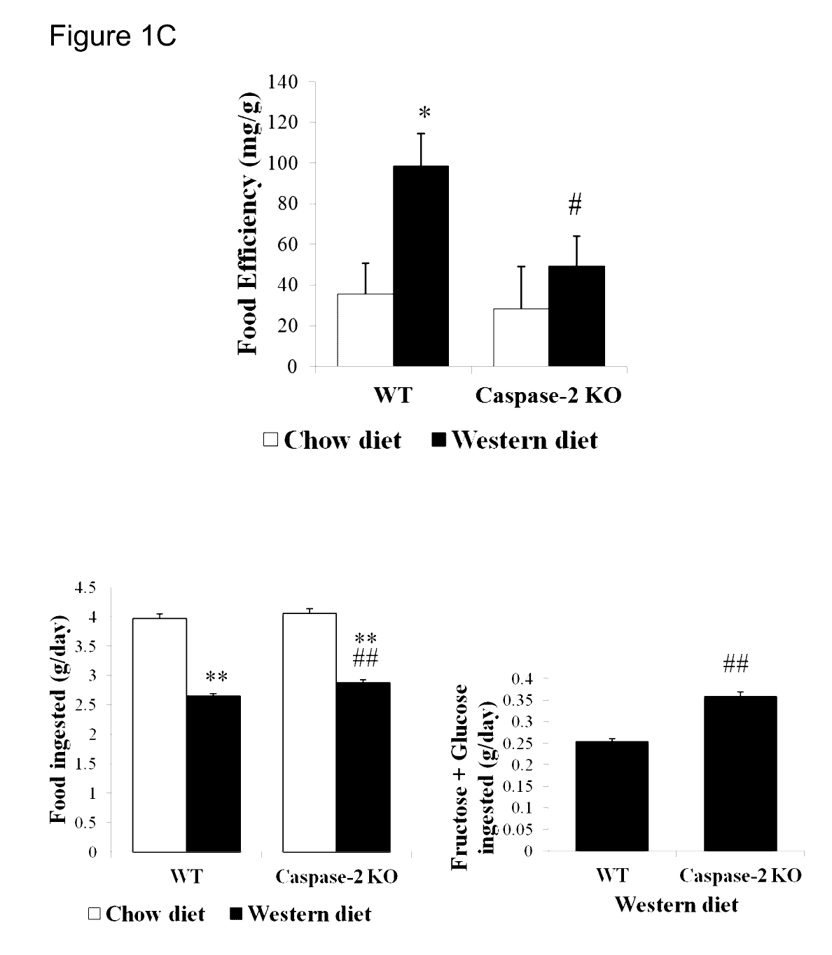

Since obesity and the MS associate with adipocyte cell death, in animal models and humans (Cancello et al., 2005; Kloting and Bluher, 2014; Strissel et al., 2007), and caspase-2 has been linked to lipotoxicity (Johnson et al., 2013; Machado et al., 2014), we aimed to study the effect of caspase-2 deficiency in corporal fat and whole body metabolism. Caspase-2 deficient mice and wild type (WT) controls were submitted to standard chow diet and high fat Western diet, for 16 weeks, starting one week after weaning. At the end of treatment, WT mice on the Western diet arm had roughly 10% more weight than the mice on chow diet. Conversely, Western diet did not induce weight gain in caspase-2 deficient mice (FIG. 1.A). Much more impressive was the difference in adiposity expansion induced by Western diet. WT mice submitted to Western diet had more than double amount of corporal fat, assessed by DXA, as compared to chow-diet fed animals, and 3-fold expansion of visceral adipose tissue. That effect on adiposity was strikingly blunted in caspase-2 deficient mice (FIG. 1.B). This is noteworthy since, more than obesity itself, the distribution of body fat with increased visceral adipose tissue in opposition to subcutaneous fat is known to be metabolically deleterious (Wajchenberg et al., 2002).

Differences in adiposity could not be explained by decreased food intake, since caspase-2 deficient mice not only did not ingest less, they even ate 8% more solid food and 40% more sugared water (with high-fructose syrup equivalent), as compared to WT mice. In fact, WT mice had higher food efficiency, that is, gained more weight per gram of food ingested (FIG. 1.C). Of note, high fructose consumption is a major determinant in the development of the MS (Kelishadi et al., 2014) and its consequences, and caspase-2 mice presented decreased central obesity despite the fact that they had higher fructose intake.

Caspase-2 Deficient Mice are Protected from Diet-Induced Glucose Metabolism Impairment

In the last week of diet, glucose and insulin tolerance tests were performed. Western diet induced glucose intolerance, in WT mice. Mice under Western diet presented a higher fasting glucose level, glucose increased more after the challenge and, most impressively, there was a delay in the recovery of glycemia in the right part of the graphic, suggesting poor insulin response to cope with hyperglycemia. Also, mice on Western diet presented a blunted response to insulin, denoting IR.

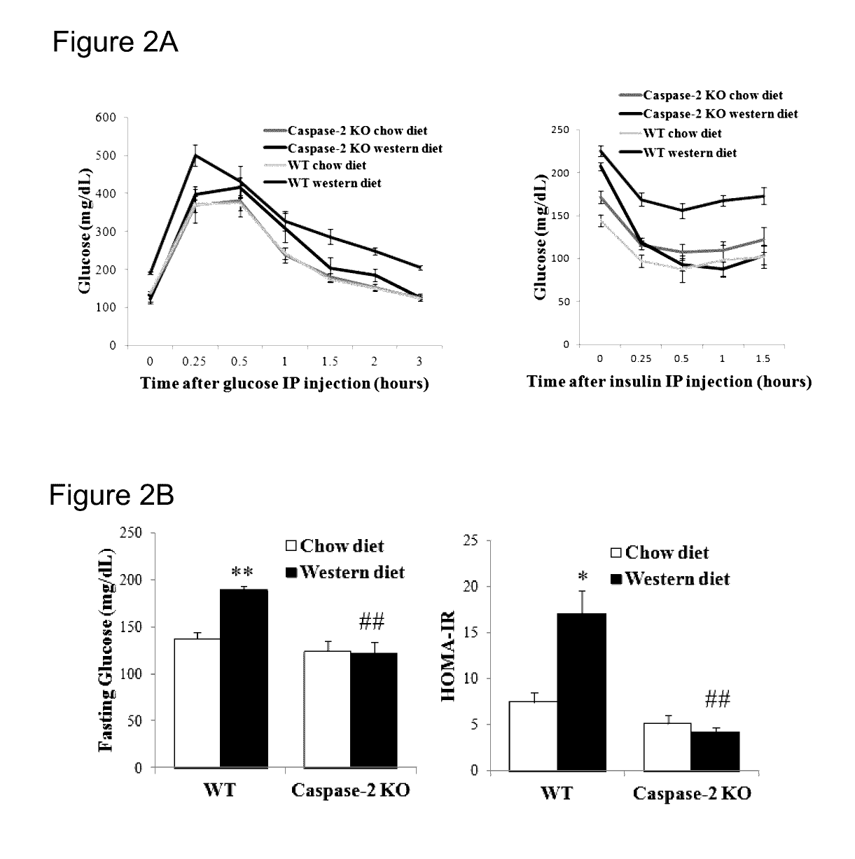

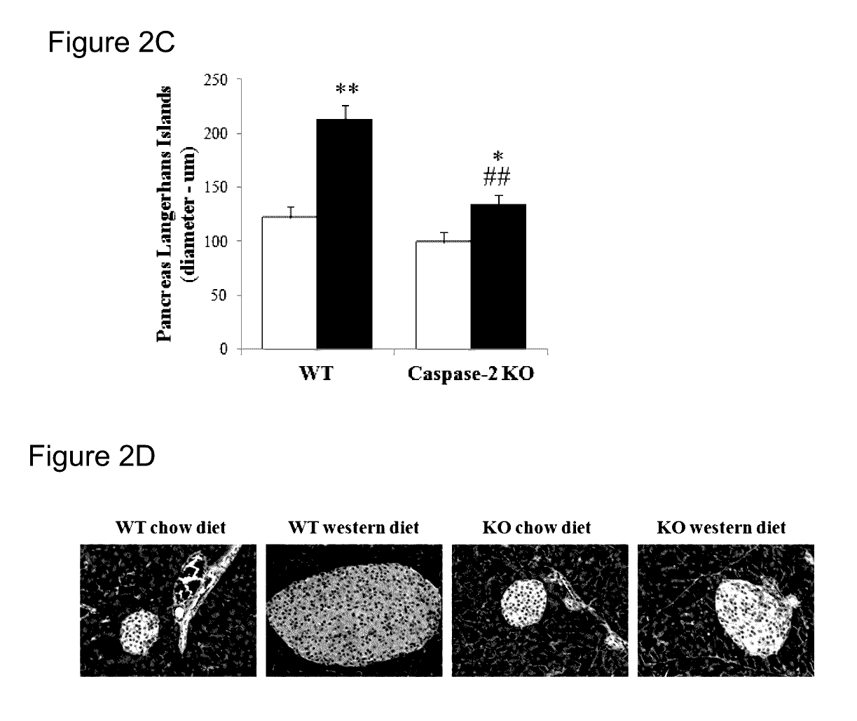

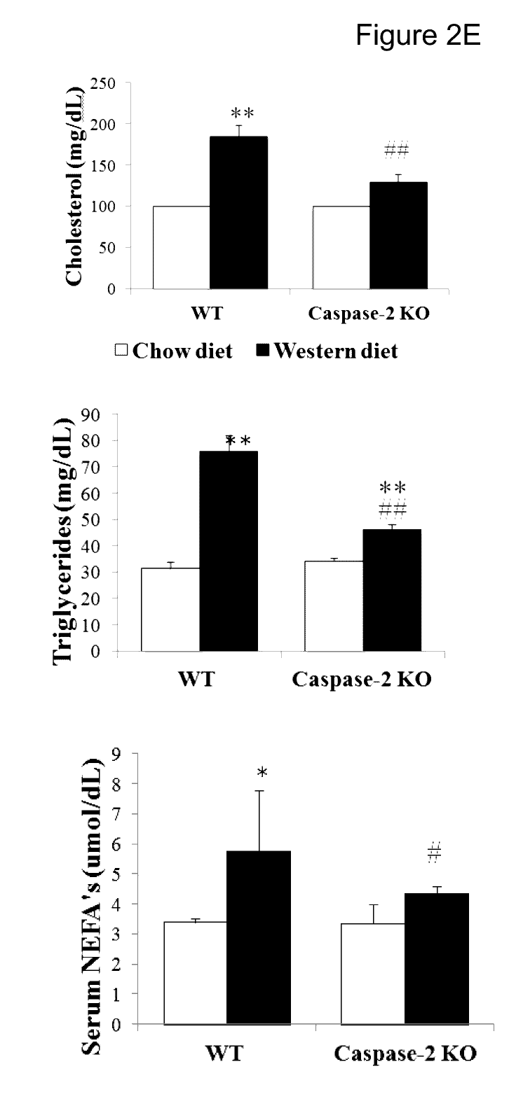

However, glycemic curves in caspase-2 deficient mice were similar to normal curves, even on Western diet (FIG. 2.A). In fact, WT submitted to Western diet developed T2DM with overnight fasting glucose levels of 200 mg/dL and HOMA-IR higher than 15, whereas caspase-2 deficient mice maintained normal glycaemia (FIG. 2.B). As expected in a mouse model of IR (Yi et al., 2013). Western diet induced hyperplasia of pancreatic Langerhans islands, response that was significantly blunted in caspase-2 deficient mice (FIGS. 2.C and D). Type 2 diabetes is described as a combination of peripheral IR that overcomes the pancreatic ability to induce adequate compensatory increase in insulin production (Kasuga, 2006).

In response to increase glucose load, pancreatic islands hypertrophy increasing insulin levels. However, if glucose challenge continues, it overcomes the hyperinsulinemic response. Eventually, insulin levels decrease as a result of overwhelming stress in β-cells. WT mice on Western diet, despite presenting extremely hyperplasia of Langerhan islands, had fasting hyperglycemia and an impaired late-response to glucose challenge, suggesting disturbed insulin secretion and hence T2DM.

Caspase-2 Deficient Mice are Protected from Diet-Induced Dyslipidemia

Western diet induced mixed dyslipidemia with increase in total cholesterol, triglycerides and non-esterified fatty acids (NEFAs). Caspase-2 deficient mice were consistently protected from dyslipidemia, in all the different lipids assessed (FIG. 2.E). The lack of increase in circulating NEFA with Western diet may be extremely relevant in the prevention of T2DM. In fact, increased circulating fatty acids induced decreased glucose transport into the muscle cells, increased liver gluconeogenesis and pancreatic β-cells dysfunction (Al-Goblan et al., 2014).

Caspase-2 Deficient Mice are Protected from Nonalcoholic Fatty Liver Disease Associated with Metabolic Syndrome

Ectopic accumulation of fat in the liver, nonalcoholic fatty liver disease (NAFLD), has been proposed as a component of the MS (Machado and Cortez-Pinto, 2014).

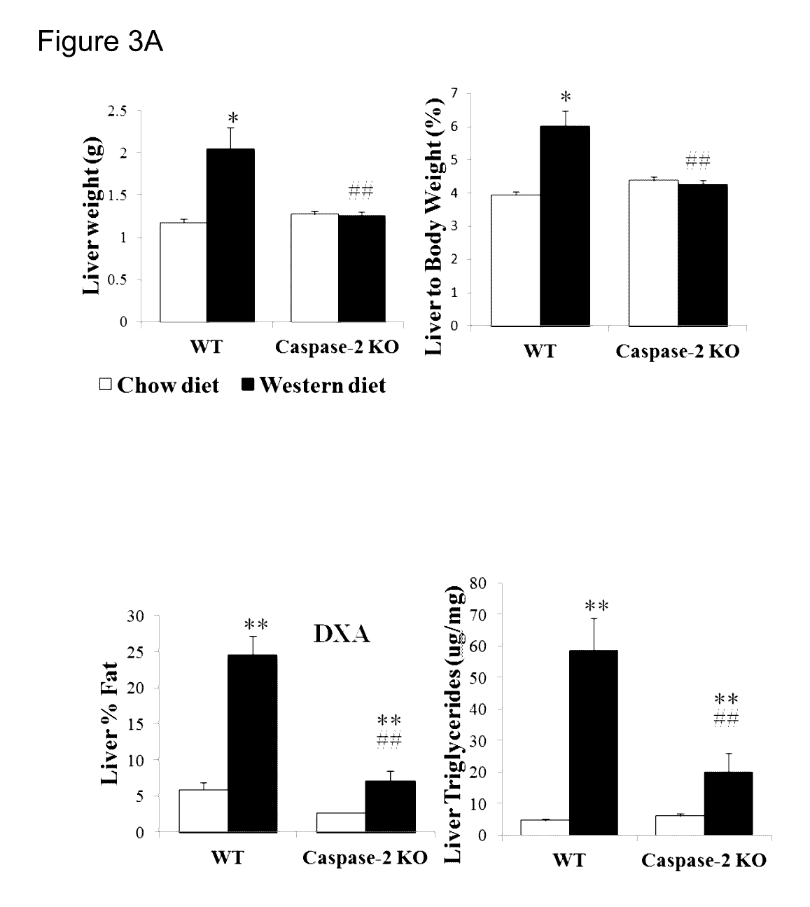

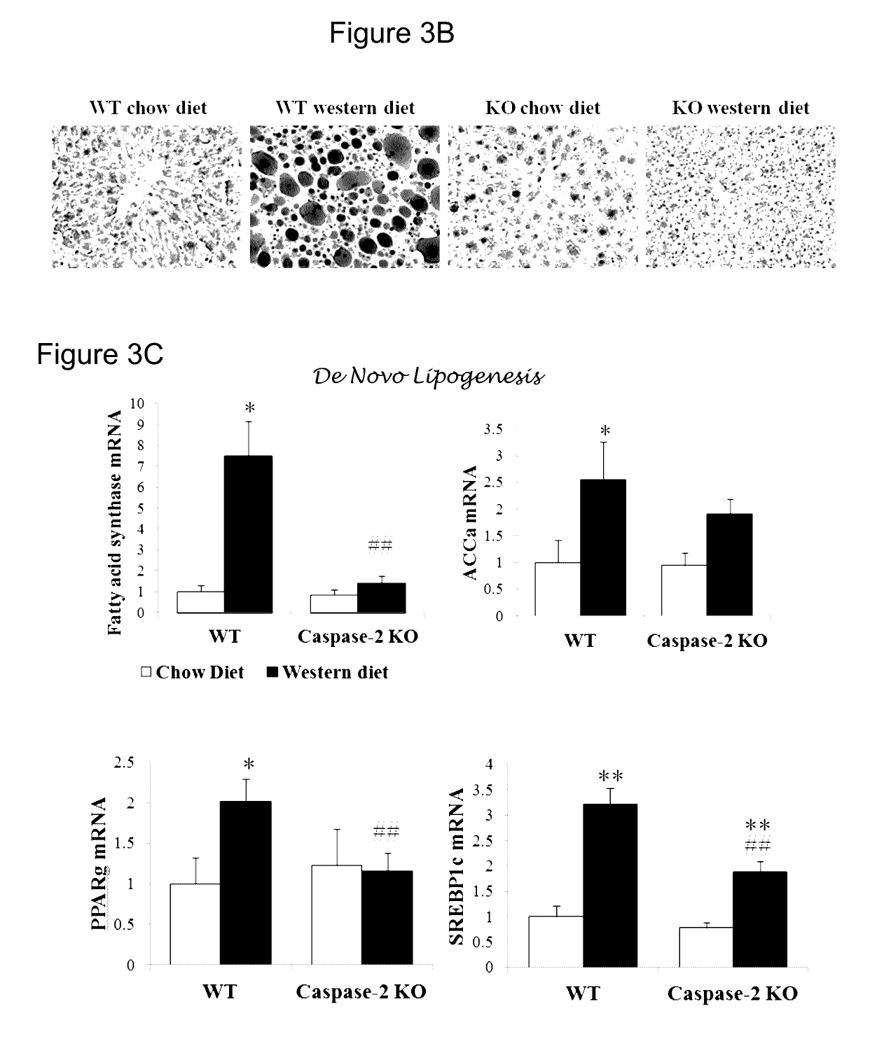

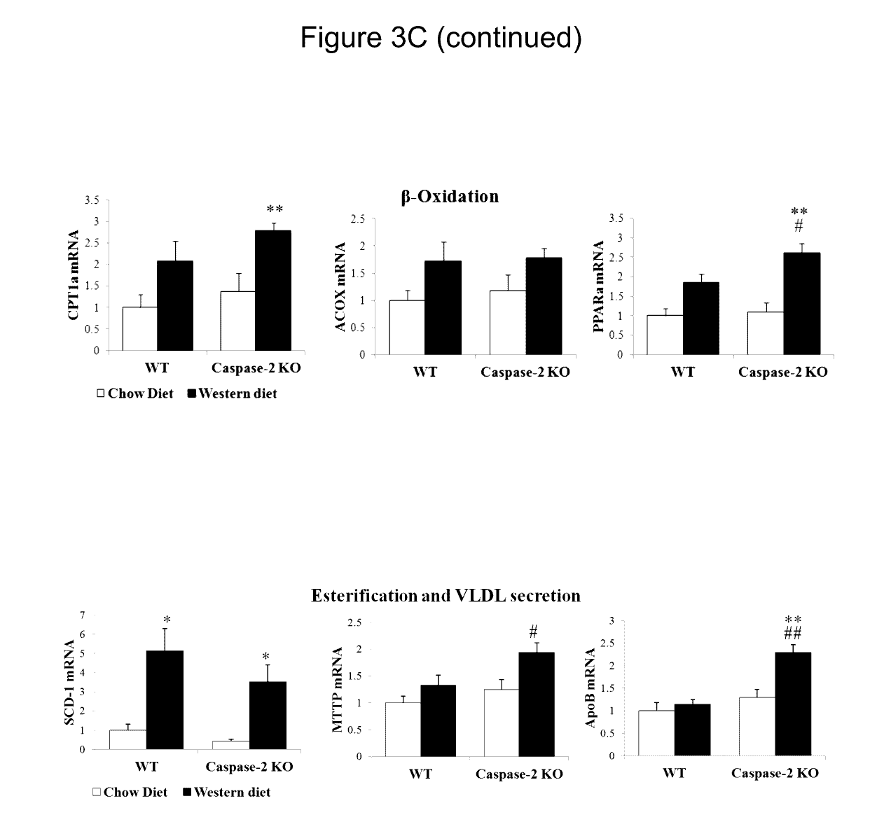

Western diet induced massive hepatomegaly in WT mice, with almost two-fold increase in liver mass and 50% increase in liver to body weight ratio. However, caspase-2 deficient mice submitted to Western diet maintained normal liver weight (FIG. 3.A). The hepatomegaly was due to a 5-6 fold increase in liver fat content, as assessed by DXA and triglycerides determination. Western diet induced a strikingly lower fat accumulation in the liver of caspase-2 mice (FIG. 3.B). Gene expression analysis of the liver showed that caspase-2 deficient mice modulate lipid metabolism towards less accumulation and higher consumption and export of fat. In fact, as compared to WT, caspase-2 deficient mice submitted to high fat diet showed lower expression of enzymes in de novo lipogenesis, and increased expression of enzymes in fatty acids β-oxidation, sterification and VLDL secretion (FIG. 3.C).

Caspase-2 Deficient Mice Reprogram Adipose Tissue, Making it More Fit to Cope with Energy Surplus

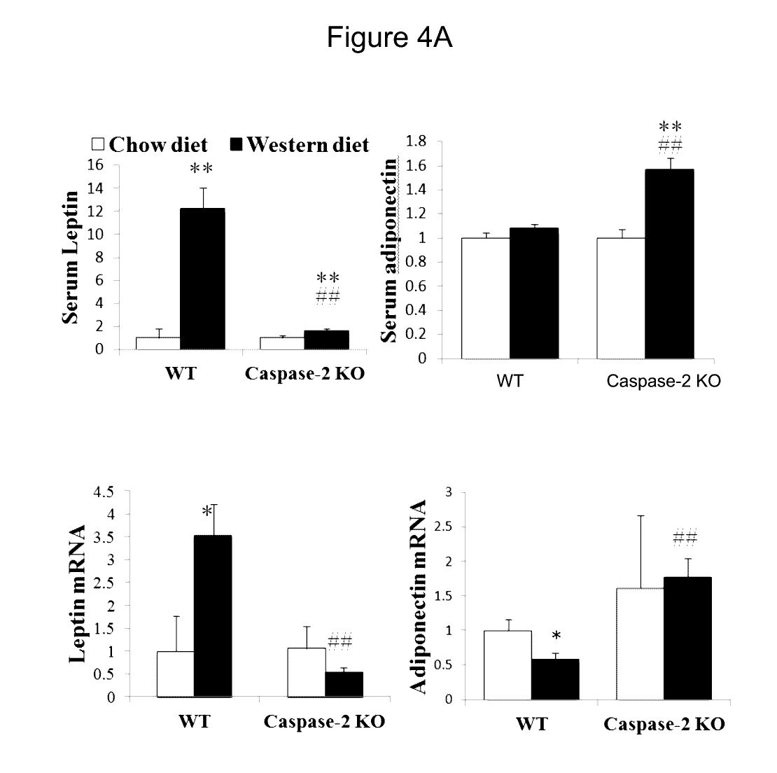

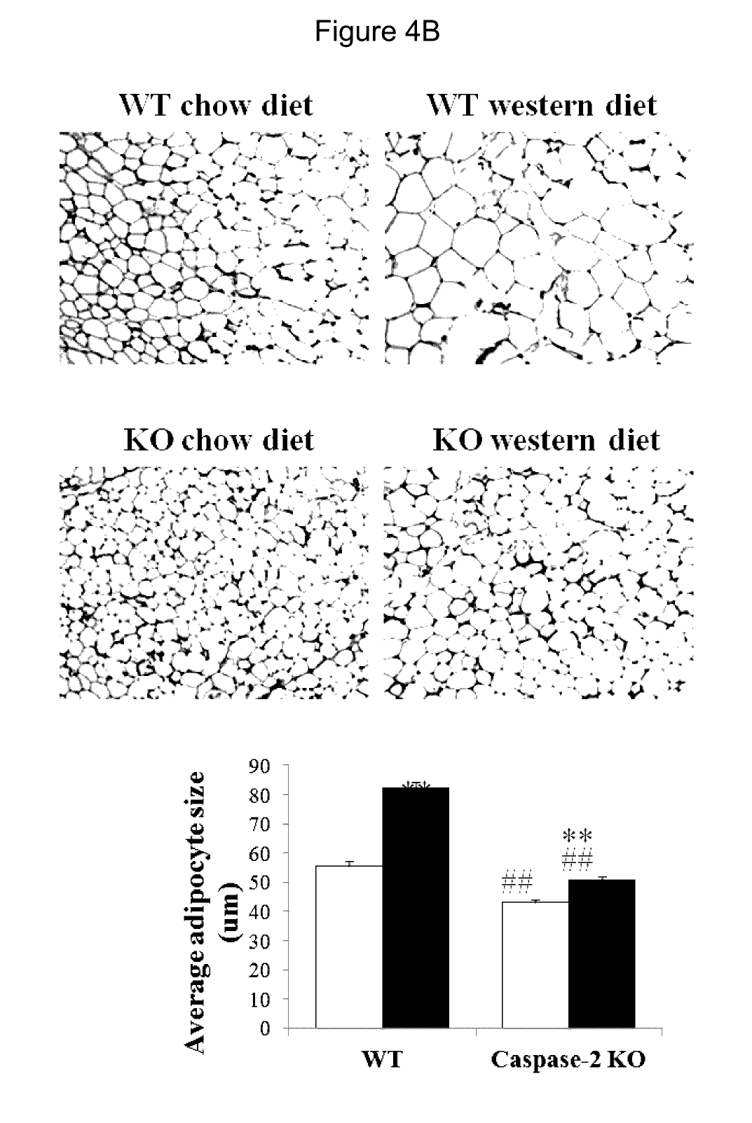

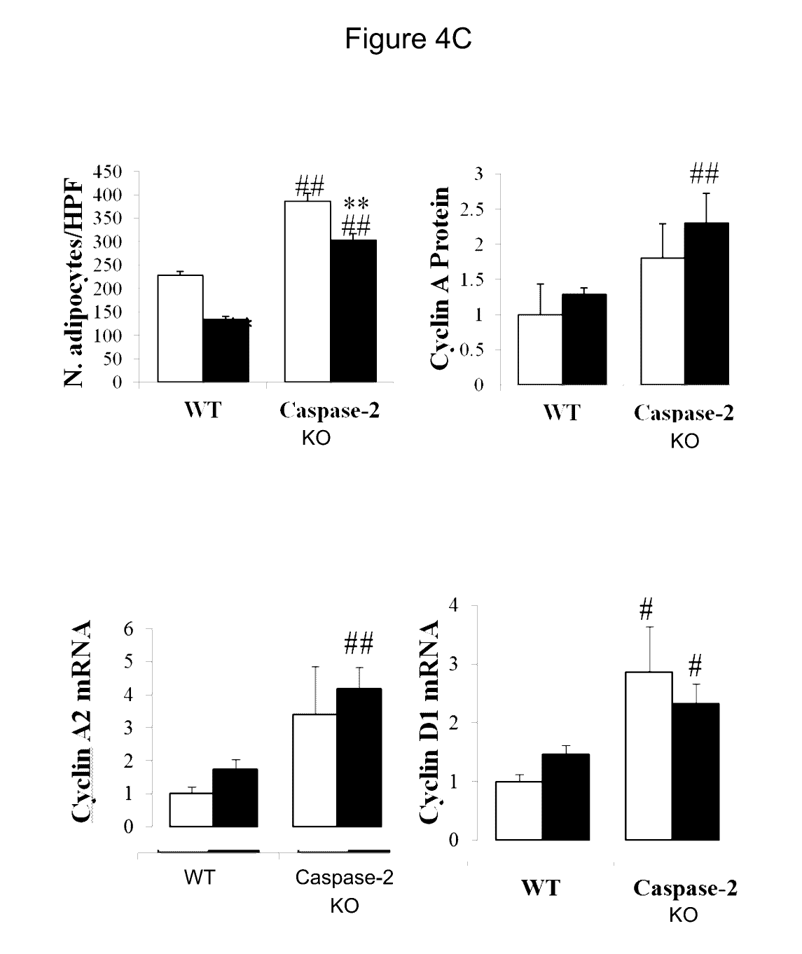

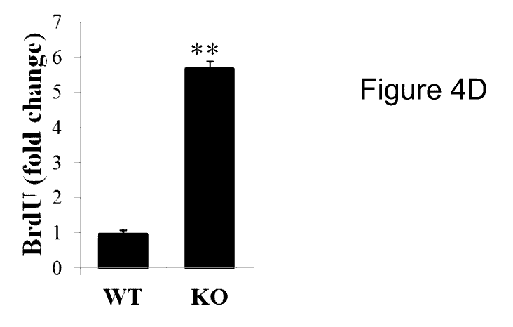

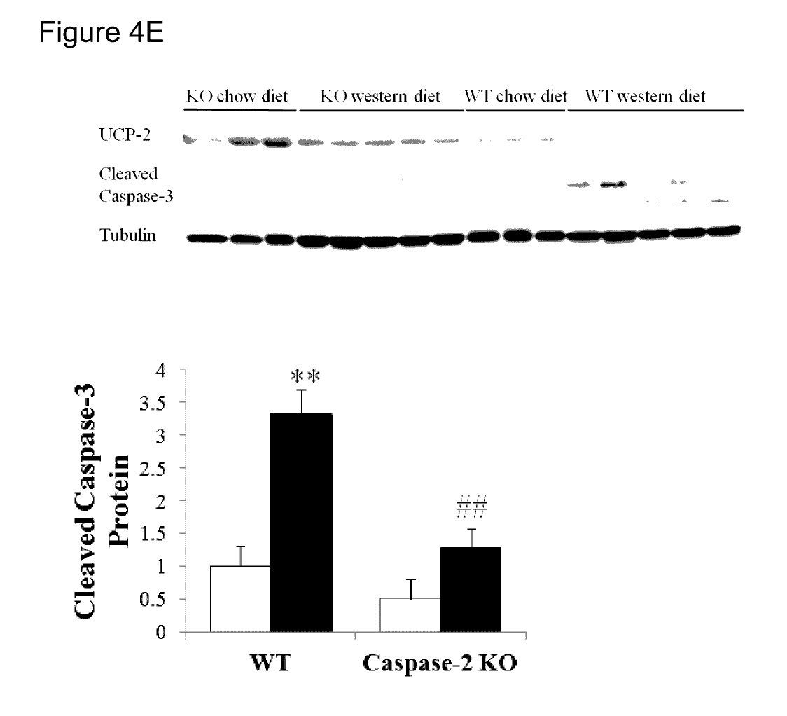

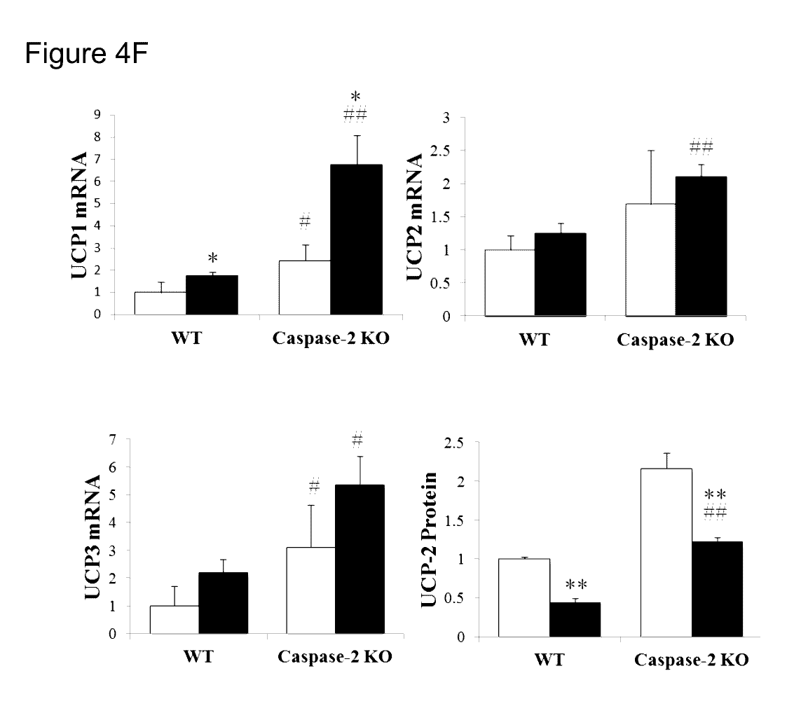

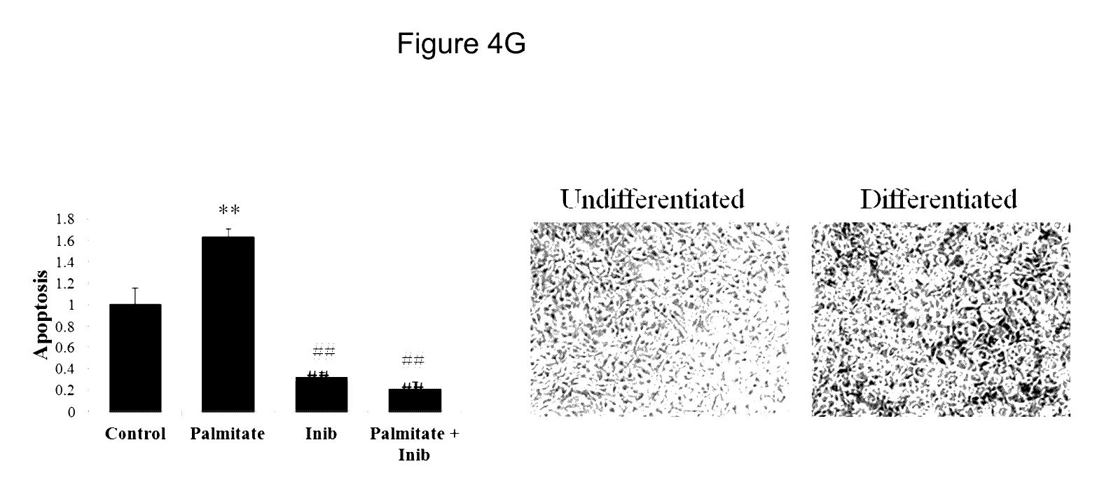

Western diet perturbs adipose tissue homeostasis leading to a deregulation in adipokine profile. It induced increased expression of leptin and reduced of adiponectin. That deregulation did not occur in caspase-2 deficient mice (FIG. 4.A). Even in chow diet, the visceral adipose tissue from caspase-2 deficient mice was fundamentally different from WT mice, with higher number of smaller adipocytes. With Western diet, the size of adipocytes markedly increased in the WT mice, but remained small in caspase-2 deficient mice (FIG. 4.B). The difference in size of adipocytes is highly relevant, since it has been shown that there is a threshold after which the risk of T2DM increases exponentially (Kloting and Bluher, 2014). Concordant with the increased number of cells, increased expression of cyclins, at mRNA and protein level, suggest increased proliferative activity in the adipose tissue of caspase-2 deficient mice (FIG. 4.C). In fact, isolated adipose stem cells from WT and caspase-2 deficient mice, showed increased proliferation in the latter (FIG. 4.D).

Also, caspase-2 deficient mice cope better with energy surplus imposed by Western diet, by decreasing the efficiency of energy obtained by fat, as suggested by an increased expression of mitochondrial uncoupling proteins (UCPs) (FIG. 4.E). UCPs decrease proton gradient across the mitochondrial inner membrane, decreasing generation of ATP from glucose and fatty acids. Manipulation of UCPs, either genetically or pharmacologically, showed a protective role in the development of obesity-related T2DM (Tao et al., 2014).

Finally, in WT mice submitted to Western diet, hyperplasia of adipocytes accompanies cell death by apoptosis as evidenced by increased visceral adipose tissue expression of cleaved caspase-3. However, there was no evidence of apoptosis activation in visceral adipose tissue from caspase-2 deficient mice (FIG. 4.F). Moreover, palmitate induced lipoapoptosis in adipocytes in cell culture, which was prevented when simultaneously treating with caspase-2 inhibitor Z-VDVAD-FMK (FIG. 4.G).

### DISCUSSION

In a mouse model of diet-induced MS, caspase-2 deficiency protects from the development of central obesity, dyslipidemia, T2DM and NAFLD, the biggest health treats of XXI century. We used a model that mimics Western diet, with 45% high fat diet enriched in saturated fat, supplemented with 0.2% cholesterol and equivalents to soft drinks. This is a non intuitive concept, of a protein involved in regulation of cell death and cell cycle having a preponderant role in whole body metabolism. In fact, regulation of body size and cell viability is a function of nutrient availability. Natural selection made us optimize the mechanisms to cope with food deprivation.

However, when we are facing energy surplus, the once adapted mechanisms became deleterious and lead to the development of the MS. Caspase-2, the most conserved caspase (Bouchier-Hayes and Green, 2012), mediates programmed cell death in models of food deprivation, through a mechanism that requires accumulation of toxic fatty acids (Johnson et al., 2013). Interestingly, caspase-2 has 2 isoforms, a long isoform (caspase-2L) that induces cell death and is expressed in most tissues, and a short isoform (caspase-2S) that can antagonize cell death, mostly expressed in the brain, heart and skeletal muscle (Kumar et al., 1997). This suggests an ancient role of caspase-2 in selecting non-vital organs to sacrifice themselves, favoring the more critical ones, in a time of imposed long periods of prolonged fasting. However, in modern times, energy surplus also leads to accumulation of toxic lipid fuels, and can in a maladaptive way induce caspase-2 activation.

We propose the following paradigm: Western lifestyle and diet leads to energy surplus and accumulation of toxic lipids in the adipocyte. The hyperplasic adipocyte activates caspase-2 that induces cell stress and death, leading to the release of toxic mediators (such as adipokines), decrease uptake of NEFA's and spill out of NEFA's from the dying cell. The net result is an increase in circulating free fatty acids and a perturbed adipokine secretion, which leads to IR and the MS. Supporting this hypothesis, caspase-2 deficient mice submitted to Western diet, when compared to WT mice in similar conditions, show less activation of apoptosis in the visceral adipose tissue, normal adipokine secretion profile, decreased levels of circulating NEFA's and protection from the development of IR/T2DM and the MS. In concordance with our findings, others have shown that caspase-2 deficient mice show resistance to age-induced glucose intolerance (Wilson et al., 2015) and induction of caspase-2 expression in adipose tissue of rats fed high-fat diet (Jobgen et al., 2009).

Unexpectedly, we found other mechanisms that may help maintain adipocyte homeostasis in response to excessive energy challenge. Visceral adipose tissue from caspase-2 deficient mice is fundamentally different from WT mice. Adipose stem cells have a higher proliferative capacity, and, as a consequence, caspase-2 deficient mice have higher number of smaller adipocytes, even when fed chow diet. Furthermore, adipocytes are equipped with more effective tools to cope with energy sources, with increased expression of mitochondrial uncoupling proteins. As a consequence, caspase-2 deficient mice have higher number of adipocytes that are able to consume more energy, and, under Western diet, do not develop stressful enlargement of adipocytes and do not expand fat mass. This is extremely important, since it has been well demonstrated that increased size of adipocyte, particularly when above a specific threshold, correlates with the development of T2DM (Kloting and Bluher, 2014).

Several mechanisms can lead to activation of caspase-2 in adipocytes overloaded with fatty acid, such as oxidative stress and endoplasmic reticulum stress (Uchibayashi et al., 2011; Upton et al., 2012). In fact, increased intracellular lipid accumulation in adipocytes activates several stress pathways including ER stress (Hotamisligil and Erbay, 2008) and oxidative stress (Jones et al., 2014). Sirtuin-1 is a member of NAD+-dependent histone deacetylase family, which acts as an important energy status sensor (Chang and Guarente, 2014). Sirtuin-1 is a known repressor of caspase-2 activity. It deacetylates 14-3-3ζ promoting its binding to caspase-2. Binding of 14-3-3ζ to caspase-2 prevents caspase-2 dephosphorylation in Ser135 and activation (Andersen et al., 2011). It has been shown that mouse models with increased sirtuin-1 expression/activity, either by genetic approaches or treatment with sirtuin-1 specific activators, are protected from NAFLD and IR when fed high fat diet (Banks et al., 2008; Feige et al., 2008).

Conversely, high fat diet in mice induces decreased levels of sirtuin-1 in adipose tissue through the cleavage, by inflammasome-dependent activation of caspase-1, of sirtuin protein (Chalkiadaki and Guarente, 2012). Similarly, human obesity associates with low levels of sirtuin-1 in visceral adipose tissue (Pedersen et al., 2008). Decrease levels/activity of sirtuin-1 in visceral adipose tissue with Western diet/obesity may be a link to caspase-2 activation and development of the MS.

Finally, it has been described that fatty acids induce lyosomal permeabilization with cathepsin activation resulting in mitochondrial dysfunction and adipocyte apoptosis. In fact, levels and activity of cathepsin D and B, were shown to be increased, in a time-dependent manner, in adipose tissue from mice fed high fat diet (Gornicka et al., 2012; Masson et al., 2011). Interestingly, in other models such as treatment of pancreatic carcinoma cell lines with Bortezomib, cathepsins are known to induce apoptosis through activation of caspase-2 (Yeung et al., 2006).

In conclusion, caspase-2 is a new potential target to address obesity and its associated comorbidities, MS, T2DM and cardiovascular disease. Those diseases are the serial killers of modern world, and to effectively treat/prevent them would have tremendous implications in human health. It is particularly appealing since we do not expect major adverse effects with a targeted approach. In fact, mice constitutively deficient in caspase-2 have a near normal phenotype.

### SEQUENCE LISTING STATEMENT

The application includes the sequence listing that is concurrently filed in computer readable form. This sequence listing is incorporated by reference herein.

### REFERENCES

All references are incorporated herein by reference in their entirety for all purposes.
Al-Goblan, A. S., Al-Alfi, M. A., and Khan, M. Z. (2014). Mechanism linking diabetes mellitus and obesity. Diabetes Metab Syndr Obes 7, 587-591.
Andersen, J. L., Thompson, J. W., Lindblom, K. R., Johnson, E. S., Yang, C. S., Lilley, L. R., Freel, C. D., Moseley, M. A., and Kornbluth, S. (2011). A biotin switch-based proteomics approach identifies 14-3-3zeta as a target of Sirt1 in the metabolic regulation of caspase-2. Mol Cell 43, 834-842.
Banks, A. S., Kon, N., Knight, C., Matsumoto, M., Gutierrez-Juarez, R., Rossetti, L., Gu, W., and Accili, D. (2008). SirT1 gain of function increases energy efficiency and prevents diabetes in mice. Cell Metab 8, 333-341.
Bouchier-Hayes, L., and Green, D. R. (2012). Caspase-2: the orphan caspase. Cell Death Differ 19, 51-57.
Cancello, R., Henegar, C., Viguerie, N., Taleb, S., Poitou, C., Rouault, C., Coupaye, M., Pelloux, V., Hugol, D., Bouillot, J. L., et al. (2005). Reduction of macrophage infiltration and chemoattractant gene expression changes in white adipose tissue of morbidly obese subjects after surgery-induced weight loss. Diabetes 54, 2277-2286.
Chalkiadaki, A., and Guarente, L. (2012). High-fat diet triggers inflammation-induced cleavage of SIRT1 in adipose tissue to promote metabolic dysfunction. Cell Metab 16, 180-188.
Chang, H. C., and Guarente, L. (2014). SIRT1 and other sirtuins in metabolism. Trends Endocrinol Metab 25, 138-145.
Cinti, S., Mitchell, G., Barbatelli, G., Murano, I., Ceresi, E., Faloia, E., Wang, S., Fortier, M., Greenberg, A. S., and Obin, M. S. (2005). Adipocyte death defines macrophage localization and function in adipose tissue of obese mice and humans. J Lipid Res 46, 2347-2355.
Feige, J. N., Lagouge, M., Canto, C., Strehle, A., Houten, S. M., Milne, J. C., Lambert, P. D., Mataki, C., Elliott, P. J., and Auwerx, J. (2008). Specific SIRT1 activation mimics low energy levels and protects against diet-induced metabolic disorders by enhancing fat oxidation. Cell Metab 8, 347-358.
Fink, T., and Zachar, V. (2011). Adipogenic Differentiation of Human Mesencgymal Stem Cells. In Mesenchymal Stem Cells Assays and Applications. M. C. Vemuri, L. G. Chase, and M. S. Rao, eds. (New York, USA: Springer).
Gornicka, A., Fettig, J., Eguchi, A., Berk, M. P., Thapaliya, S., Dixon, L. J., and Feldstein, A. E. (2012). Adipocyte hypertrophy is associated with lysosomal permeability both in vivo and in vitro: role in adipose tissue inflammation. Am J Physiol Endocrinol Metab 303, E597-606.
Grundy, S. M., Brewer, H. B., Jr., Cleeman, J. I., Smith, S. C., Jr., Lenfant, C., National Heart, L., Blood, I., and American Heart, A. (2004). Definition of metabolic syndrome: report of the National Heart, Lung, and Blood Institute/American Heart Association conference on scientific issues related to definition. Arterioscler Thromb Vasc Biol 24, e13-18.
Hotamisligil, G. S., and Erbay, E. (2008). Nutrient sensing and inflammation in metabolic diseases. Nat Rev Immunol 8, 923-934.
Jobgen, W., Fu, W. J., Gao, H., Li, P., Meininger, C. J., Smith, S. B., Spencer, T. E., and Wu, G. (2009). High fat feeding and dietary L-arginine supplementation differentially regulate gene expression in rat white adipose tissue. Amino acids 37, 187-198.
Johnson, E. S., Lindblom, K. R., Robeson, A., Stevens, R. D., Ilkayeva, O. R., Newgard, C. B., Kornbluth, S., and Andersen, J. L. (2013). Metabolomic profiling reveals a role for caspase-2 in lipoapoptosis. J Biol Chem 288, 14463-14475.
Jones, D. A., Prior, S. L., Barry, J. D., Caplin, S., Baxter, J. N., and Stephens, J. W. (2014). Changes in markers of oxidative stress and DNA damage in human visceral adipose tissue from subjects with obesity and type 2 diabetes. Diabetes Res Clin Pract 106, 627-633.
Kasuga, M. (2006). Insulin resistance and pancreatic beta cell failure. J Clin Invest 116, 1756-1760.
Kelishadi, R., Mansourian, M., and Heidari-Beni, M. (2014). Association of fructose consumption and components of metabolic syndrome in human studies: a systematic review and meta-analysis. Nutrition 30, 503-510.
Kloting, N., and Bluher, M. (2014). Adipocyte dysfunction, inflammation and metabolic syndrome. Rev Endocr Metab Disord 15, 277-287.
Kumar, S., Kinoshita, M., Dorstyn, L., and Noda, M. (1997). Origin, expression and possible functions of the two alternatively spliced forms of the mouse Nedd2 mRNA. Cell Death Differ 4, 378-387.
Machado, M. V., and Cortez-Pinto, H. (2014). Management of fatty liver disease with the metabolic syndrome. Expert Rev Gastroenterol Hepatol 8, 487-500.
Machado, M. V., Michelotti, G. A., Pereira, T. D., Boursier, J., Kruger, L., Swiderska-Syn, M., Karaca, G., Xie, G., Guy, C. D., Bohinc, B., et al. (2014). Reduced lipoapoptosis, hedgehog pathway activation and fibrosis in caspase-2 deficient mice with non-alcoholic steatohepatitis. Gut.
Machado et al. (2016). Caspase-2 promotes obesity, the metabolic syndrome and nonalcoholic fatty liver disease. Cell Death and Disease 7, e2096; doi:10.1038/cddis.2016.19.
Machado et al. (2015). Vitamin B5 and N-Acetylcysteine in Nonalcoholic Steatohepatitis: A Preclinical Study in a Dietary Mouse Model. Dig Dis Sci; doi:10.1007/s10620-015-3871-x.
Masson, O., Prebois, C., Derocq, D., Meulle, A., Dray, C., Daviaud, D., Quilliot, D., Valet, P., Muller, C., and Liaudet-Coopman, E. (2011). Cathepsin-D, a key protease in breast cancer, is up-regulated in obese mouse and human adipose tissue, and controls adipogenesis. PloS one 6, e16452.
Michelotti, G. A., Xie, G., Swiderska, M., Choi, S. S., Karaca, G., Kruger, L., Premont, R., Yang, L., Syn, W. K., Metzger, D., et al. (2013). Smoothened is a master regulator of adult liver repair. J Clin Invest 123, 2380-2394.
Nguyen, D. M., and El-Serag, H. B. (2010). The epidemiology of obesity. Gastroenterol Clin North Am 39, 1-7.
Pedersen, S. B., Olholm, J., Paulsen, S. K., Bennetzen, M. F., and Richelsen, B. (2008). Low Sirt1 expression, which is upregulated by fasting, in human adipose tissue from obese women. Int J Obes 32, 1250-1255.
Schweizer A., Roschitzki-Voser H., Amstutz P., Briand C., Gulotti-Georgieva M., Prenosil E., Binz H. K., Capitani G., Baici A., Pluckthun A., Grutter M. G. (2007). Inhibition of caspase-2 by designed ankyrin repeat protein: specificity, structure, and inhibition mechanism. Structure 15, 625-636.
Strissel, K. J., Stancheva, Z., Miyoshi, H., Perfield, J. W., 2nd, DeFuria, J., Jick, Z., Greenberg, A. S., and Obin, M. S. (2007). Adipocyte death, adipose tissue remodeling, and obesity complications. Diabetes 56, 2910-2918.
Tao, H., Zhang, Y., Zeng, X., Shulman, G. I., and Jin, S. (2014). Niclosamide ethanolamine-induced mild mitochondrial uncoupling improves diabetic symptoms in mice. Nat Med 20, 1263-1269.
Tsigos, C., Hainer, V., Basdevant, A., Finer, N., Fried, M., Mathus-Vliegen, E., Micic, D., Maislos, M., Roman, G., Schutz, Y., et al. (2008). Management of obesity in adults: European clinical practice guidelines. Obes Facts 1, 106-116.
Uchibayashi, R., Tsuruma, K., Inokuchi, Y., Shimazawa, M., and Hara, H. (2011). Involvement of Bid and caspase-2 in endoplasmic reticulum stress- and oxidative stress-induced retinal ganglion cell death. J Neurosci Res 89, 1783-1794.
Upton, J. P., Wang, L., Han, D., Wang, E. S., Huskey, N. E., Lim, L., Truitt, M., McManus, M. T., Ruggero, D., Goga, A., et al. (2012). IRE1alpha cleaves select microRNAs during ER stress to derepress translation of proapoptotic Caspase-2. Science 338, 818-822.
Wajchenberg, B. L., Giannella-Neto, D., da Silva, M. E., and Santos, R. F. (2002). Depot-specific hormonal characteristics of subcutaneous and visceral adipose tissue and their relation to the metabolic syndrome. Horm Metab Res 34, 616-621.
WHOM, C. (2013). Obesity and overweight. Genebre Switzerland.
Wilson, C. H., Shalini, S., Filipovska, A., Richman, T. R., Davies, S., Martin, S. D., McGee, S. L., Puccini, J., Nikolic, A., Dorstyn, L., et al. (2015). Age-related proteostasis and metabolic alterations in Caspase-2-deficient mice. Cell Death Dis 6, e1597.
Yeung, B. H., Huang, D. C., and Sinicrope, F. A. (2006). PS-341 (bortezomib) induces lysosomal cathepsin B release and a caspase-2-dependent mitochondrial permeabilization and apoptosis in human pancreatic cancer cells. J Biol Chem 281, 11923-11932.
Yi, P., Park, J. S., and Melton, D. A. (2013). Betatrophin: a hormone that controls pancreatic beta cell proliferation. Cell 153, 747-758.
Zebisch, K., Voigt, V., Wabitsch, M., and Brandsch, M. (2012). Protocol for effective differentiation of 3T3-L1 cells to adipocytes. Anal Biochem 425, 88-90.
Zhang, Y., Padalecki S. S., Chaudhuri A. R., De Waal E., Goins B. A., Grubbs B., Ikeno Y., Richardson A., Mundy G. R., Herman B. (2007). Caspase-2 deficiency enhances aging-related traits in mice. Mech Ageing Dev 128, 213-221.

We claim:

1. A method of treating or protecting a patient from a condition selected from the group consisting of obesity, insulin resistance, hyperinsulinemia, type 2 diabetes mellitus, dyslipidemia, and nonalcoholic fatty liver disease comprising the step of treating a patient with an effective amount of caspase-2 inhibitor such that the condition is improved.

2. The method of claim 1 wherein the caspase-2 inhibitor does not significantly inhibit caspases other than caspase-2.

3. The method of claim 1 wherein the condition is nonalcoholic fatty liver disease.

4. The method of claim 3 wherein the treatment is for a period of at least one year.

5. The method of claim 1 wherein the treatment comprises the additional step of evaluating the success of the treatment by evaluating the patient before and during treatment.

6. The method of claim 5 wherein the additional step comprises an evaluation selected from the group consisting of evaluation of weight and BMI, glucose tolerance test, the need to take anti-diabetic therapy, serum lipid profile, and the need to take anti-hypertension therapy.

7. The method of claim 5 wherein the condition is liver disease and the evaluation is selected from examination of serum liver enzymes and ultrasound monitoring.

8. A method of reprogramming adipose tissue to decrease adipocyte size, increase adipocyte viability, increase adipocyte proliferation, increase adipocyte metabolic uncoupling or modulate gene expression in the liver comprising the step of treating a patient with an effective amount of caspase-2 inhibitor such that the patient's adipocytes are modified in a manner more consistent with a healthier individual.

9. The method of claim 8, wherein the evaluation of efficacy of the treatment comprises evaluation of adipose tissue function by determination visceral adipose tissue size and plasma levels of adipokines leptin and/or adiponectin, expecting normalization of serum levels to indicate effectiveness.
